(19) United States
(12) Patent Application Publication
(10) Pub. No.: US 2026/0096976 A1
(43) Pub. Date: Apr. 9, 2026

(54) AGENT FOR REPIGMENTATION OF KERATIN FIBERS, IN PARTICULAR HUMAN HAIR

(71) Applicant: DR. KURT WOLFF GMBH & CO. KG, Bielefeld (DE)

(72) Inventors: Erik SCHULZE ZUR WIESCHE, Bielefeld (DE); Miriam HAGENSTEIN, Bielefeld (DE)

(73) Assignee: DR. KURT WOLFF GMBH & CO. KG, Bielefeld (DE)

(21) Appl. No.: 19/114,001

(22) PCT Filed: Sep. 22, 2023
(86) PCT No.: PCT/EP2023/076198

§ 371 Date: Mar. 21, 2025

(30) Foreign Application Priority Data
Sep. 22, 2022 (EP) 22197130.2

(51) Int. Cl. A61K 8/49; A61K 8/41; A61Q 5/02; A61Q 5/10

(52) U.S. Cl. CPC A61K 8/492; A61K 8/418; A61K 8/4953; A61Q 5/10; A61K 2800/4324; A61K 2800/592; A61Q 5/02

(57) ABSTRACT

The present invention relates to a cosmetic formulation containing a coloring agent precursor selected from the group that consists of indole derivatives and indoline derivatives, and a xanthine derivative. The invention also relates to the use of the cosmetic formulation for repigmentation of keratin fibers, in particular human hair.

The present invention relates to a cosmetic formulation containing a dye precursor selected from the group consisting of indole derivatives and indoline derivatives, and a xanthine derivative. The invention further relates to the use of the cosmetic formulation for the repigmentation of keratin fibers, in particular human hair.

A person's hair color depends on the amount of the melanins eumelanin and pheomelanin, which are contained in the fibrous layer of the hair and are produced by melanocytes in the hair follicles. As part of the normal ageing process, but also due to certain states of diseases, stress, vitamin and mineral deficiencies or excessive alcohol or nicotine consumption, melanin production can decrease, resulting in hypopigmentation. Such hair appears white or colorless to the observer. The optical impression of gray hair results from the mixture of pigmented and pigmentless hair.

The average life cycle of a hair is three to seven years, after which the hair falls out and a new hair grows in its place. Gradually, the proportion of white or pigmentless hair predominates and the hair turns gray.

The change in hair color and in particular the pigmentation or repigmentation of gray hair is an important part of modern cosmetics. In particular, good fastness properties and good gray coverage as well as long-lasting coloration are desirable.

For permanent, intensive dyeing with good fastness properties and good gray coverage, oxidation dyes are conventionally used. Such dyes usually contain oxidation dye precursors, so-called developer components and coupler components, which form the actual dyes under the influence of oxidizing agents, such as hydrogen peroxide, or of atmospheric oxygen with each other or under coupling with one or more coupler components.

However, the use of oxidative dyes is still associated with impairment or damage to the hair and in particular its surface structure.

Alternatively, precursors of the natural hair dye melanin can be applied to the hair, which form practically nature-identical dyes as part of oxidative processes in the hair. The coloration can be carried out with atmospheric oxygen as the only oxidizing agent, so that no further oxidizing agents are required. Such a method using 5,6-dihydroxyindoline as a dye precursor is described, for example, in EP 0 530 229 B1.

In contrast, direct dyes, also known as substantive dyes, are used for temporary dyeing. These are dye molecules that are applied directly to the hair and do not need an oxidative process to create the color. In comparison to oxidative hair dyeing, the dyes obtained with direct dyes are less durable and wash out more quickly. Colorings with direct dyes usually remain on the hair for a period of between 5 and 20 hairwashes.

WO 99/66890 describes a dyeing agent for dyeing keratin fibers containing a dye precursor, which may be an indoline or indole derivative, e.g. a derivative of 5,6-dihydroxyindoline or 5,6-dihydroxyindole, as well as an amino acid or an oligopeptide. The color can be formed by means of air oxidation.

DE 10 2018 127 182 A1 relates to a two-component system for artificial hair coloring containing, separately from one another, an anhydrous carrier medium which comprises an alkane, a fatty alcohol and an alcohol, as well as an aqueous phase which contains hydrogen peroxide. An pforganosilicon compound from the group of silanes may be contained for the protection and care of the hair.

DE 10 2007 038 484 A1 describes a hair treatment agent for protection against external influences, whereby the hair treatment agent contains bacterial ferment(s) from Thermus thermophilus.

WO 2005/007615 A1 relates to 2-(amino- or substituted amino)-5-(substituted oxymethyl)-phenol compounds and compositions for the oxidative dyeing of keratin fibers.

However, with the formulations known in the art, it is not possible to achieve sufficiently satisfactory results in terms of quality, intensity and duration of the color performance while at the same time impairing or damaging the hair structure as little as possible.

Thus, with the precursors of the natural hair dye melanin the resulting color bodies are formed with a significant time delay and the color shades cannot be adequately controlled. In addition, the intermediate shades are often unnaturally violet. Another disadvantage is the unpigmented hairline that appears as the hair grows longer.

In summary, there is still room for improvement, particularly in terms of fastness properties, gray coverage and the duration of the dyeing process.

Based on this, the problem underlying the present invention was to provide a cosmetic formulation which is suitable for pigmenting or repigmenting keratin fibers, in particular human hair, and in particular offers improved fastness properties of the pigmented or repigmented keratin fibers as well as improved gray coverage. Furthermore, this cosmetic formulation should have improved properties in terms of quality, intensity and duration of the coloring performance compared to formulations known from the prior art.

This problem was surprisingly solved by the formulation according to claim 1 and its use according to claim 11. Preferred embodiments are shown in the dependent claims.

According to the invention, the problem is solved by providing a cosmetic formulation containing a dye precursor selected from the group consisting of indole derivatives and indoline derivatives, and a xanthine derivative.

Surprisingly, it was found that the contrast to the regrowing hair (hairline) is significantly lower, as the natural pigment formation in the hair root is stimulated, while at the same time darkening the hair that has already grown out.

Overall, the hair gradually becomes darker, whereby the color effect gradually builds up, gray hair is clearly covered and the overall impression of the hair color appears significantly darker.

Thus, the cosmetic formulation according to the invention achieves good gray coverage and excellent fastness properties whilst maintaining the color performance for a long time and reducing the graying of the hairline.

The xanthine derivative contained in the formulation according to the invention, together with the dye precursor, effects stimulation of the keratinocytes in the hair roots so that they produce more natural pigment again. Furthermore, the xanthine derivative causes the hair to remain on the head for longer. This delays the loss of still pigmented hair and also contributes to the natural repigmentation of already pigment-poor or pigmentless hair. In the context of the present invention, the terms pigmentation and repigmentation are used interchangeably.

The dye precursor contained in the formulation according to the invention, which is an indole derivative and/or indoline derivative, is preferably an indole derivative and/or indoline derivative having a hydroxy or amino group, preferably as a substituent on the six-membered ring. These groups can carry further substituents, e.g. in the form of an etherification or esterification of the hydroxy group or an alkylation of the amino group. Compounds with two of these groups, in particular two hydroxy groups, one or both of which may be etherified or esterified, are particularly preferred.

In a preferred embodiment of the invention, the dye precursor is an indole derivative.

According to the invention, the dye precursor is preferably a derivative of 5,6-dihydroxyindole of formula (I),

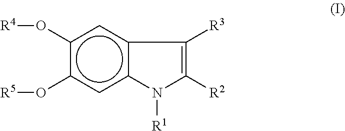

wherein independently of each other,
R¹ is hydrogen, a C₁-C₄-alkyl group or a C₁-C₄-hydroxyalkyl group,
R² is hydrogen or a —COOH group, wherein the —COOH group can also be present as a salt with a physiologically tolerated cation,
R³ is hydrogen or a C₁-C₄-alkyl group,
R⁴ is hydrogen, a C₁-C₄-alkyl group, an amino group or a group —CO—R⁶ in which R⁶ is a C₁-C₄-alkyl group, and
R⁵ is one of the groups mentioned under R⁴, or a physiologically tolerated salt of these compounds with an organic or inorganic acid.

Preferred representatives according to the invention are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole.

In a particularly preferred embodiment, the compound of formula (I) is selected from 5,6-dihydroxyindole, N-methyl-5,6-dihydroxyindole and physiologically tolerated salts thereof.

Particularly preferably, the dye precursor used in the formulation according to the invention is 5,6-dihydroxyindole.

In a further preferred embodiment of the invention, the dye precursor is an indoline derivative.

According to the invention, the dye precursor is preferably a derivative of 5,6-dihydroxyindoline of formula (II),

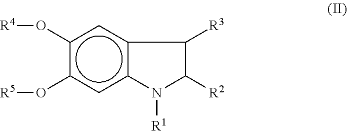

wherein, independently of each other,
R¹ is hydrogen, a C₁-C₄-alkyl group or a C₁-C₄-hydroxyalkyl group,
R² is hydrogen or a —COOH group, wherein the —COOH group can also be present as a salt with a physiologically tolerated cation,
R³ is hydrogen or a C₁-C₄-alkyl group,
R⁴ is hydrogen, a C₁-C₄-alkyl group, an amino group or a group —CO—R⁶ in which R⁶ is a C₁-C₄-alkyl group, and
R⁵ is one of the groups mentioned under R⁴, or a physiologically tolerated salt of these compounds with an organic or inorganic acid.

Preferred representatives according to the invention are 5,6-dihydroxyindoline, N-methyl-5,6-dihydroxyindoline, N-ethyl-5,6-dihydroxyindole.

In a particularly preferred embodiment, the compound of formula (II) is selected from 5,6-dihydroxyindoline, N-methyl-5,6-dihydroxyindoline and physiologically tolerated salts thereof.

Particularly preferably, the dye precursor used in the formulation according to the invention is 5,6-dihydroxyindoline.

In another particularly preferred embodiment, the dye precursor used in the formulation according to the invention is dihydroxyindoline hydrobromide (2,3-dihydro-1H-indole-5,6-diol hydrobromide).

The indole and indoline derivatives contained in the agents according to the invention can be used both as free bases and in the form of their physiologically tolerated salts with inorganic or organic acids, e.g. hydrochlorides, sulphates and hydrobromides.

In the formulation according to the invention the indole or indoline derivative is usually present in an amount of 0.001-5% by weight, preferably 0.005-2% by weight, preferably 0.01-2% by weight, preferably 0.01-1.5% by weight., preferably in an amount of about 0.05% by weight, 0.1% by weight, 0.2% by weight, 0.3% by weight, 0.4% by weight, 0.5% by weight, 0.6% by weight, 0.7% by weight, 0.8% by weight, 0.9% by weight, 1.0% by weight, 1.5% by weight, in each case based on the total weight of the formulation.

In a particularly preferred embodiment, the dye precursor is an indole derivative, preferably 5,6-dihydroxyindole, which is present in the formulation according to the invention in an amount of 0.01-2% by weight, preferably 0.05-0.6% by weight, preferably in an amount of 0.08-0.5% by weight, preferably in an amount of about 0.1-0.5% by weight, in each case based on the total weight of the formulation.

In a further particularly preferred embodiment, the dye precursor is an indoline derivative, preferably dihydroxyindoline hydrobromide (2,3-dihydro-1H-indole-5,6-diol hydrobromide), wherein it is present in the formulation according to the invention in an amount of 0.01-4.0% by weight, preferably 0.05-2.0% by weight, preferably in an amount of about 0.08-1.0% by weight, preferably in an amount of about 0.1-0.5% by weight, in each case based on the total weight of the formulation.

The formulation according to the invention may also contain more than one indole or indoline derivative or mixtures of indole and indoline derivatives.

The pigment is formed from the dye precursor, i.e. the indole or indoline derivative, by reaction with atmospheric oxygen; no additional oxidizing agent is required.

The xanthine derivative contained in the cosmetic formulation, together with the dye precursor, leads in particular to the keratinocytes in the hair roots being stimulated to produce more natural pigment again. In addition, the xanthine derivative causes the hair to remain on the head for longer, which delays the loss of hair that is still pigmented and also contributes to the natural repigmentation of hair that is already low in pigment or lacking pigment.

The preferred xanthine derivative according to the invention is caffeine.

The IUPAC name of caffeine is 1,3,7-trimethyl-3,7-dihydro-1H-purine-2,6-dione. Alternatively, caffeine is also known as 1,3,7-trimethylxanthine. Caffeine is represented by the following chemical formula (III).

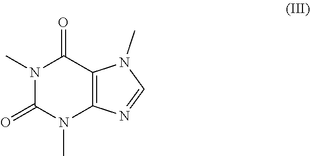

[symbol]

a methylxanthine alkaloid belonging to the class of methylxanthine. It is a bitter crystalline substance, can be regarded as a purine derivative and is chemically related to the adenine and guanine bases of deoxyribonucleic acids and ribonucleic acid.

In the formulation according to the invention, the xanthine derivative is usually present in an amount of 0.0001-10% by weight, preferably 0.01-5% by weight, preferably 0.05-2% by weight, preferably 0.1-1.5% by weight, preferably in an amount of about 0.0001% by weight, 0.05% by weight, 0.1% by weight, 0.2% by weight, 0.3% by weight, 0.5% by weight, 0.7% by weight, 1.0% by weight, 1.3% by weight, 1.4% by weight, 1.5% by weight, in each case based on the total weight of the formulation.

In a preferred embodiment, the formulations according to the invention further comprise at least one direct dye.

Surprisingly, it was found that the color effect of a formulation that also contains a direct dye in addition to the dye precursor of the indole or indoline type and the xanthine derivative is more intense than would have been expected based on the color effects of the individual components, dye precursor of the indole or indoline type and direct dye. The darkening effects (repigmentation) are synergistically enhanced by the combination with direct dyes in terms of the speed of color penetration and color depth.

Thus, in the presence of a combination of a dye precursor of the indole or indoline type and a direct dye, a synergistic effect occurs. Consequently, the cosmetic formulation according to the invention comprising a dye precursor of the indole or indoline type, a xanthine derivative and a direct dye advantageously achieves a particularly intense color effect, with excellent gray coverage and excellent fastness properties, i.e. a highly natural shade with a long-lasting dyeing result.

As mentioned at the beginning, direct dyes are dye molecules that are applied directly to the hair and do not require an oxidative process to form the color.

Preferred direct dyes comprise nitrophenylene diamines, nitroamino phenols, azo dyes, anthraquinones and indophenols and are preferably selected from the group of dyes known by the international designations or trade names HC Yellow 2, HC Yellow 4, HC Yellow 5, HC Yellow 6, HC Yellow 12, Acid Yellow 1, Acid Yellow 10, Acid Yellow 23, Acid Yellow 36, Basic Yellow 57, Basic Yellow 87, HC Orange 1, Disperse Orange 3, Acid Orange 7, Basic Orange 31, HC Red 1, HC Red 3, HC Red 10, HC Red 11, HC Red 13, HC Red BN, Acid Red 33, Acid Red 52, Pigment Red 57:1, Basic Red 51, Basic Red 76, HC Blue 2, HC Blue 12, HC Blue 16, Disperse Blue 3, Acid Blue 7, Basic Blue 99, Acid Green 50, HC Violet 1, Disperse Violet 1, Disperse Violet 4, Acid Violet 43, Disperse Black 9, Acid Black 1, Acid Black 52, Basic Brown 16 and Basic Brown 17 as well as 1,4-diamino-2-nitrobenzene, 2-amino-4-nitrophenol, 1,4-bis-(β-hydroxyethyl)-amino-2-nitrobenzene, 3-nitro-4-(β-hydroxyethyl)-aminophenol, 2-(2-hydroxyethyl)amino-4,6-dinitrophenol, 1-(2′-hydroxyethyl)amino-4-methyl-2-nitrobenzene, 1-amino-4-(2-hydroxyethyl)-amino-5-chloro-2-nitrobenzene, 4-amino-3-nitrophenol, 1-(2-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, hydroxyethyl-2-nitro-toluidine, picramic acid and its salts, 2-amino-6-chloro-4-nitrophenol, 4-ethylamino-3-nitrobenzoic acid and 2-chloro-6-ethylamino-1-hydroxy-4-nitrobenzene.

Commonly, the direct dye is contained in the formulation according to the present invention in an amount of 0.001-5% by weight, preferably 0.05-3% by weight, preferably 0.1-2% by weight, preferably 0.1-1.2% by weight, preferably in an amount of about 0.1% by weight, 0.2% by weight, 0.3% by weight, 0.4% by weight, 0.5% by weight, 0.6% by weight, 0.7% by weight, 0.8% by weight, 0.9% by weight, 1.0% by weight, 1.1% by weight, 1.2% by weight, 1.3% by weight, 1.4% by weight, 1.5% by weight, 1.6% by weight, 1.7% by weight, 1.8% by weight, 1.9% by weight, 2.0% by weight, in each case based on the total weight of the formulation.

In a preferred embodiment, the direct dye is selected from the group consisting of 2-amino-6-chloro-4-nitrophenol, 1,4-bis-(β-hydroxyethyl)-amino-2-nitrobenzene, Basic Yellow 57, Basic Red 76, Basic Brown 16, Basic Brown 17, Basic Blue 99, HC Blue 2, HC Blue 12, HC Blue 16 and Acid Violet 43.

In a particularly preferred embodiment of the invention, the direct dye is HC Blue 2.

In a particularly preferred embodiment, the formulation according to the invention contains 5,6-dihydroxyindole as dye precursor and caffeine as xanthine derivative. Here, 5,6-dihydroxyindole is contained in an amount of 0.05-0.6% by weight and caffeine is contained in an amount of 0.8-1.2% by weight, in each case based on the total weight of the formulation.

In a further particularly preferred embodiment, the formulation according to the invention contains 5,6-dihydroxyindole as dye precursor, caffeine as xanthine derivative and further HC Blue 2 as direct dye. Here, 5,6-dihydroxyindole is contained in an amount of 0.05-0.6% by weight, caffeine is contained in an amount of 0.8-1.2% by weight and the direct dye is contained in an amount of 0.1-1.2% by weight, in each case based on the total weight of the formulation.

Furthermore, the cosmetic formulation according to the invention may contain any additives known in such preparations (i.e. active ingredients, additives and excipients).

In one embodiment, the cosmetic formulation comprises a surfactant. The surfactant may be an anionic, nonionic, cationic or zwitterionic surfactant. Preferably, the surfactant is an anionic or nonionic surfactant, in particular an anionic or nonionic surfactant that is as mild as possible (i.e, particularly well tolerated by the skin). According to the invention, nonionic surfactants are in particular used because of their very good emulsifying properties and their excellent skin care properties. Anionic surfactants are preferred because they have a particularly high cleaning performance. They are therefore particularly suitable for cleansing formulations such as shampoos. Cationic surfactants have excellent hair care properties and are used according to the invention in particular in hair care formulations such as conditioners, shampoos and treatments.

The formulation according to the invention contains surfactants preferably in an amount of from 2 to 40% by weight, in particular from 5 to 30% by weight, preferably from 7 to 20% by weight, particularly preferably from 10 to 17% by weight, in each case based on the total weight of the formulation. Suitable amounts of surfactant are: 8% by weight; 9% by weight; 10% by weight; 11% by weight; 12% by weight; 13% by weight; 14% by weight; 15% by weight; 16% by weight; 17% by weight; 18% by weight; 19% by weight; 20% by weight; 21% by weight; 22% by weight; 23% by weight; 24% by weight; 25% by weight, in each case based on the total weight of the formulation. Particularly preferably, the cosmetic formulation of the invention contains one or more anionic surfactants in an amount of from 0.1 to 20% by weight, preferably from 1 to 17% by weight and particularly preferably from 5 to 15% by weight, in each case based on the total weight of the formulation. Suitable amounts of anionic surfactant are: 1% by weight; 2% by weight; 3% by weight; 4% by weight; 5% by weight; 6% by weight; 7% by weight; 8% by weight; 9% by weight; 10% by weight; 11% by weight; 12% by weight; 13% by weight; 14% by weight; 15% by weight; 16% by weight; 17% by weight; 18% by weight, 19% by weight, 20% by weight, in each case based on the total weight of the formulation. In these quantities, the surfactants have a particularly high cleansing performance and are extremely well tolerated by the skin, scalp and hair.

The surfactants of the present invention are described, inter alia, in the book “Surfactants and interfacial phenomena”, by Milton Rosen and Joy Kunjappu, John Wiley & Sons, Inc., 2012, 4th edition.

In a preferred embodiment, the surfactant is an anionic surfactant selected from alkyl carboxylates, alkyl sulfonates, alkyl sulfates, alkyl ether sulfates, alkyl phosphates, alkyl sarcosinates, alkyl taurates, amino acid surfactants and mixtures thereof. Particularly preferred the surfactant is selected from alkyl carboxylates, alkyl sulphates, alkyl sarcosinates, alkyl taurates, alkyl glutamate, such as sodium cocoyl glutamate/disodium cocoyl glutamate, alkyl gycinate, alkyl alaninate, such as sodium cocoyl alaninate and mixtures thereof. Likewise, fatty alcohol polyglycerol ether sulphates, monoglyceride sulphates, mono- and/or dialkyl sulphosuccinates, fatty acid isethionates and α-olefin sulphonates are preferred because of their cleaning performance.

Alkyl carboxylates have the generic formula RCO₂M, alkyl sulfates have the generic formula ROSO₃M, alkyl sarcosinates have the generic formula RC(O)N(CH₃)CH₂CO₂M, and alkyl taurates have the generic formula RC(O)N(CH₃)CH₂CH₂SO₃M, wherein R each is a C₄-C₂₆-alkyl or C₄-C₂₆-alkenyl an M is a water-soluble cation such as ammonium, sodium or potassium. Preferably, M is a sodium cation. Preferably, R is a C₁₂-C₁₆-alkyl or a C₁₂-C₁₈-alkyl.

In one embodiment, the surfactant is a non-ionic surfactant (also referred to as a non-ionic emulsifier). Non-limiting examples comprise glycerol fatty acid esters, fatty alcohols, polyoxyethylene ethers of one or more fatty alcohols, alkoxylated fatty acid alkyl esters, polyglycerol ethers of fatty alcohols, polyglycerol esters of fatty acids, polyethylene glycol and/or polypropylene glycol ethers, fatty acid amides, alkylphenol polyglycol ethers, amine oxides and alkyl polyglucosides.

In one embodiment, the surfactant is selected from the group of glycerol fatty acid esters, fatty alcohols, polyoxyethylene ethers of one or more fatty alcohols, polyglycerol ethers of fatty alcohols, polyglycerol esters of fatty acids and mixtures thereof.

In the present invention, the term “glycerol fatty acid ester” refers to a glycerol mono- or glycerol di-fatty acid ester. Glycerol di-fatty acid esters have the formula R³—COO—(CH₂CH(OH)CH₂)—OOR⁴ or R³—COO—(CH₂CH(OOR⁴)CH₂)—OH. Glycerol mono-fatty acid esters have the formula R³—COO—(CH₂CH(OH)CH₂)—OH or HO—(CH₂CH(OOR³)CH₂)—OH. Hereby, R³ and R⁴ are independently selected from C₆-C₂₈-alkyl and C₆-C₂₈-alkenyl. Glycerol mono-fatty acid esters contain a glycerol group which is linked to a single fatty acid via an ester bond. Examples are glycerol monostearate, glycerol monobehenate, glycerol monocaprylate, glycerol monocaprate and glycerol monolaurate.

Fatty alcohols (saturated) are compounds of the formula H₃C—(CH₂)ₙ—CH₂—OH, where n is an integer between 4 and 20, preferably an integer between 6 and 16.

Polyoxyethylene ethers are compounds of the formula R⁵ (OC₂H₃)ₙOH, wherein R⁵ is selected from C₆-C₂₈-alkyl, C₆-C₂₈-alkenyl, substituted and unsubstituted phenoxy groups; and n is an integer greater than 1. Preferably, the polyoxyethylene ether of one or more fatty alcohols is selected from the group of steareth-2, steareth-21, macrogol cetostearyl ether 12, ceteareth-25, macrogol cetostearyl ether 20 and mixtures of the aforementioned compounds. Even more preferably, the polyoxyethylene ether is a compound selected from the group of ceteareth-25, macrogol cetostearyl ether 20 and mixtures of the aforementioned compounds.

The term “polyglycerol ether of fatty alcohols” refers to a compound of the formula R⁶O—(C₃H₆O₂)ₙ—H, wherein R⁶ is a branched or linear C₆-C₂₈-alkyl or C₆-C₂₈-alkenyl and n is an integer greater than 1, preferably an integer from 2 to 10. It is preferred that the formulation contains 0.01 to 15.0% by weight, 0.1 to 10.0% by weight or 1 to 5.0% by weight of polyglycerol ether.

The term “polyglycerol ester of fatty acids” refers to compounds containing both a polyglycerol unit and at least one C₆-C₂₆-alkyl or C₆-C₂₆-alkenyl carboxylic acid unit. These compounds may have the formula R⁷—R⁸—(C₃H₆O₂)ₙ—H, wherein R⁷ is a C₆-C₂₆-alkanoate or C₆-C₂₆-alkenoate residue and R⁸ is a suitable linking molecule or a direct bond. Thus, the polyglycerol moiety and the C₍₆₎-C₂₆-alkyl or C₆-C₂₆-alkenyl carboxylic acid moiety may be directly linked by an ester bond or may contain a linker moiety linking these two moieties with each other. Non-limiting examples of this group are polyglyceryl-3-methylglucose distearate, polyglycerol polycrinoleate, polyglyceryl dimerate isostearate, polyglyceryl-2-laurate, polyglyceryl-2-sesquiisostearate, polyglyceryl-3-distearate (Cremophor GS 32), polyglyceryl-3-oleate, polyglyceryl-3-methylglycose distearate, polyglyceryl-4-caprate (polyglycerol caprate T2010190), polyglyceryl-4-diisostearate/polyhydroxystearate/sebacate (Isolan GPS) and polyglyceryl-4-isostearate.

In one embodiment, the surfactant comprises a cationic surfactant, such as a quaternary surfactant. Quaternary surfactants contain at least one N atom that is covalently linked to 4 alkyl or aryl groups. This leads to a positive charge, regardless of the pH value. Alkyl betaine, alkyl amidopropyl betaine and alkyl amidopropyl hydroxysulphaine are advantageous. The cationic surfactants used according to the invention can also preferably be selected from the group of quaternary ammonium compounds, in particular benzyltrialkylammonium chlorides or bromides, such as benzyldimethylstearylammonium chloride, and alkyltrialkylammonium salts, for example cetyltrimethylammonium chloride or bromide, alkyldimethylhydroxyethylammonium chlorides or bromides, dialkyldimethylammonium chlorides or bromides, alkylamidethyltrimethylammonium ether sulphates, alkylpyridinium salts, for example lauryl- or cetylpyrimidinium chloride, imidazoline derivatives and compounds with cationic character such as amine oxides, for example alkyldimethylamine oxides or alkylaminoethyldimethylamine oxides. Using cetyltrimethylammonium salts is particularly advantageous.

In a further embodiment, the formulation according to the invention contains at least one additive. Additives which are usually used in shampoos, conditioners and emulsions for treating the skin, scalp and hair, are preferred. The at least one additive may be present in a proportion of from 0.01% by weight to 12.0% by weight, more preferably from 0.25% by weight to 10.0% by weight, in particular from 1.0% by weight to 7.0% by weight.

The at least one additive may further be selected from the group consisting of hair conditioning agents, moisturizers, preservatives, stabilizers, fragrances, antioxidants, rheology modifiers, thickening agents, care agents, dyes, pearlizing agents, brightening agents, solvents, firming compounds, anti-dandruff agents, vitamins, alkalizing agents or pH adjusters and combinations thereof.

Hair conditioning agents can reduce static charges of the hair by neutralizing the electrical charge on its surface. Examples of hair conditioning agents are quaternary ammonium compounds.

Moisturizers, also known as moisturizing agents or superfatting agents, are lipophilic substances that can prevent a disruptive effect on the epidermal barrier function. Examples of refatting agents are lanolin, squalene, liquid paraffin, vegetable oils, silicones and cetyl palmitate.

Preservatives are substances that are used for preservation by killing and/or inhibiting the growth of microorganisms that decompose the formulation. Preferably, the preservatives may be selected from the group consisting of benzoic acid, benzoic acid derivatives, sorbic acid, sorbic acid derivatives, salicylic acid, salicylic acid derivatives, phenoxyethanol, parabens and combinations thereof. In a preferred embodiment, sodium benzoate and/or potassium sorbate are used as preservatives in the formulation according to the invention. Sodium benzoate releases benzoic acid and potassium sorbate releases sorbic acid in a slightly acidic environment. Both acids are said to have an antimicrobial effect.

Stabilizers can protect light-sensitive components against radiation and are preferably UV absorbers such as benzophenone derivatives.

The addition of fragrances can give the formulation a pleasant smell. Examples are the perfumes known to experts.

An antioxidant or antioxidizing agent is a chemical compound that slows down or completely prevents oxidation of other components in the formulation according to the invention. Examples of antioxidants include citric acid, ascorbic acid, sodium sulfite and butylated hydroxyanisole.

Rheology modifiers and thickeners can help to improve the application properties of the formulation according to the invention. The addition of common salt (sodium chloride) can be considered as a rheology modifier and thickener. By adding common salt, the flowability of the formulation according to the invention can be influenced within certain limits and adjusted to the required level. Naturally occurring gelling agents can also be used as thickeners, which are preferably selected from agar, xanthan gum, cellulose and/or cellulose derivatives or alginic acid.

In the context of the application, care agents are to be understood as substances that provide care for the hair and/or scalp. Hydrolyzed wheat protein, panthenol and allantoin have a nourishing effect on the scalp and hair. Hydrolyzed wheat protein has mainly moisturizing properties.

As further dyes, the formulation according to the invention may contain colorants, for example to give the formulation an optically appealing color. The formulation according to the invention may contain, for example, CI 47005 as a suitable colorant.

The solvent used can be a solvent or solvent mixture commonly used by a person skilled in the art in this field. Preferred solvents are ethanol or butylene glycol, in particular 1,4-butylene glycol, propylene glycol and isopropyl alcohol. Ethanol or propylene glycol is preferred. These solvents can preferably be present in an amount of 0.1 to 70% by weight in the formulation according to the invention. Most preferably, they are present in an amount of 0.1 to 5.0% by weight.

Firming compounds are preferably selected from the group consisting of waxes, synthetic polymers and mixtures thereof. Suitable firming compounds are known to the skilled person and are described, for example, in Goddard, E. Desmond; Gruber, James V. (1999), “Principles of polymer science and technology in cosmetics and personal care”, Series: Cosmetic science and technology series: v. 22., New York (Marcel Dekker, INC.); Schrader, Karlheinz (1989), “Grundlagen und Rezepturen der Kosmetika” (2nd edition), chapter 3.7, Heidelberg (Hüthig); and Scott, Richard (2017), “ingredients focus: cosmetic waxes and butters”, Personal Care Europe 10 (3), 46-47.

Anti-dandruff agents are used to control excessive formation of dandruff on the scalp. Suitable anti-dandruff agents are known to the person skilled in the art and can be found, for example, in Trüeb, R. M. (2009). “Kopfschuppen erfolgreich behandeln.” Akt Dermatol 35 (01/02): 19-24; Trueb, R. M. (2007). “Shampoos: ingredients, efficacy and adverse effects.” J. Dtsch. Dermatol. Ges. 5 (5): 356-365; Sanfilippo, A. and J. English (2006). “An overview of medicated shampoos used in dandruff treatment.” P AND T 31 (7): 396; as well as Futterer, E. (1981). “Evaluation of efficacy of antidandruff agents.” J Soc Cosmet Chem 32:327-338 described.

Vitamins and minerals such as niacinamide (vitamin B3), vitamin E or zinc are used to achieve a positive effect on the growth and repair of damaged hair. Suitable vitamins and minerals are known to the person skilled in the art and are described, for example, in Pietrzik K, Golly I, Loew D, Handbuch Vitamine, Elsevier GmbH, Urban & Fischer Verlag, Munich 2008; H. Lautenschläger, Vitamine in der Kosmetik, medical Beauty Forum 2020 (3), 14-17; as well as Martini M. C., Chivot M., Peyrefitte G., Lehrbuch Kosmetik: Grundlagen-Grundstoffe-Grundtechniken, Hogrefe AG, Bern 2001.

Alkalizing agents or pH adjusters are used to adjust the pH value of the formulation to a desired range. Suitable alkalizing agents or pH adjusters comprise, among others, sodium hydroxide, monoethanolamine 0.1-5%, phosphoric acid and citric acid.

Examples of compositions are shown in the following table:

| Component | Quantity in % by weight |
|---|---|
| Aqua [water] | ad 100 |
| Sodium lauryl ether sulfate | 5-15 |
| Sodium myreth sulfate | 0-5 |
| Sodium cocoamphoacetate | 0-5 |
| Laureth-2 | 0-5 |
| Cocoamidopropyl betaine | 0-5 |
| Disodium lauryl sulfosuccinate | 0-5 |
| Sodium lauroyl glutamate | 0-5 |
| Panthenol | 0-5 |
| Caffeine | 0.1-5 |
| Sodium chloride | 0-5 |
| Phenoxyethanol | 0-5 |
| Wheat protein hydrolyzate | 0-3 |
| Cocoglucoside | 0-3 |
| Glyceryl oleate | 0-3 |
| Phosphoric acid | 0-3 |
| Sodium benzoate | 0-3 |
| PEG-120 methyl glucose dioleate | 0-3 |
| Potassium sorbate | 0-3 |
| HC Blue 2 | 0.1-2 |
| Allantoin | 0-3 |
| PEG-40 hydrogenated castor oil | 0-3 |
| Menthol | 0-2 |
| Polyquaternium-7 | 0-2 |
| Sodium hydroxide | 0-2 |
| Disodium EDTA | 0-2 |
| Dihydroxyindole | 0.01-2 |
| Tocopherol | 0-2 |
| Citric acid | 0-2 |
| Niacinamide | 0-2 |
| Zinc PCA | 0-2 |
| Dye/colorant | 0-2 |

According to the invention, the composition is applied topically. A topical application means external application, in particular a local, external application. Compositions according to the invention are preferably hair care products (i.e. treatment compositions such as shampoos, conditioners or treatments).

In a preferred embodiment, the formulation according to the invention is present as a shampoo, conditioner, treatment or color treatment, in particular as shampoo or color treatment.

The cosmetic formulation of the present invention is particularly suitable for pigmentation and/or repigmentation of keratin fibers.

Accordingly, in a further embodiment, the present invention relates to the use of the cosmetic formulation containing a dye precursor selected from the group consisting of indoline derivatives and indole derivatives, and a xanthine derivative as bioactive agent for pigmenting and/or repigmenting keratin fibers.

For the purposes of the present invention, “keratin fibers” means hair, in particular human hair. In this context, the term “human hair” in particular comprises hair on the head and facial hair.

In a preferred embodiment, the present invention relates to the use of the cosmetic formulation for pigmentation and/or repigmentation of human hair.

The formulation is preferably distributed in the wet or dry hair and rinsed out after a dwell time of between 1 and 30 minutes.

The cosmetic formulation according to the invention for the pigmentation and/or repigmentation of keratin fibers, in particular human hair, has the effect that the hair is permanently colored in a slow and gentle manner and at the same time the keratinocytes in the hair roots are stimulated so that they produce more natural pigment again.

Thus, in a further embodiment, the invention relates to the use of the cosmetic formulation for pigmentation and/or repigmentation of human hair, wherein the hair is permanently colored in a slow and gentle manner and at the same time the keratinocytes in the hair roots are stimulated so that they produce more natural pigment again.

In a further embodiment, the invention relates to a method for pigmenting and/or repigmenting keratin fibers, in particular human hair, wherein the cosmetic formulation according to the invention is applied to the hair and then the coloration takes place. The coloring is effected solely by atmospheric oxygen; no further oxidizing agent is required.

The following formulations are intended to illustrate the invention, however, they do not intend to limit it to the specific examples.

### DESCRIPTION OF THE FIGURES

FIG. 1 Comparative photos of strands washed with Example Formulation 2 (D1/D2) and Comparative Formulation (E1/E2), respectively

FIG. 2 Comparison of the color preview for the used two shampoos Example Formulation 2 and Comparative Formulation, respectively

FIG. 3 Change in color depth when using the shampoos Example Formulation 2 and Comparative Formulation, respectively, as absolute values dL*

FIG. 4 Total color change dE* when using the shampoos Example Formulation 2 and Comparative Formulation, respectively

FIG. 5 Change in color depth when washing on with color shampoo (5 washes) and washing out with neutral formulation (14 washes), respectively

FIG. 6 Change in color depth when washing on with color shampoo (10 washes) and washing out with neutral formulation (15 washes), respectively

FIG. 7 Change in color depth when washing on with color shampoo (15 washes) and washing out with neutral formulation (15 washes), respectively

FIG. 8 Comparison of the color change for the two shampoos used on a single strand after 15 wash-on cycles, followed by 15 wash-out cycles

FIG. 9 Comparison of the color preview for the used shampoos Example Formulation 1 and Example Formulation 2 and Comparative Formulation, respectively, on a single strand in each case with 15 wash-on cycles

FIG. 10 Change in color depth when using the shampoos Example Formulation 1 and Example Formulation 2, respectively, and Comparative Formulation, respectively, as absolute values dL*

FIG. 11 Total color change dE* when using the shampoos Example Formulation 1 and Example Formulation 2, respectively, and Comparative Formulation, respectively

FIG. 12 Comparison of the color preview for the used shampoos Example Formulation 6, and Example Formulation 7, respectively, and Example Formulation 8, respectively, on a single strand at 15 wash-on cycles in each case

FIG. 13 Change in color depth when using the shampoos Example Formulation 6 or Example Formulation 7 or Example Formulation 8 as absolute values dL*

FIG. 14 Total color change dE* when using the shampoos Example Formulation 6 and Example Formulation 7, respectively, and Example Formulation 8, respectively

FIG. 15 Comparative photos of strands washed with Example Formulation 6 (“DHI-B”), Example Formulation 7 (“Combi-B”) and Example Formulation 8 (“Blue-B”), respectively, using strand B as an example

### EXPERIMENTAL PART

### Formulations

The following formulations have been prepared by means known to a person skilled in the art.

### Comparative Formulation

The Comparative Formulation contains caffeine as xanthine derivative and HC Blue 2 as direct dye.

| Component | Quantity in % by weight |
|---|---|
| Aqua [water] | ad 100 |
| Sodium lauryl ether sulfate | 11.5 |
| Sodium cocoamphoacetate | 3 |
| Cocamidopropyl betaine | 1.7 |
| Caffeine | 1.1 |
| Panthenol | 0.92 |
| Sodium chloride | 1 |
| Phenoxyethanol | 0.7 |
| Cocoglucoside | 0.5 |
| Glyceryl oleate | 0.5 |
| Phosphoric acid | 0.4 |
| PEG-120 methyl glucose dioleate | 0.3 |
| HC Blue 2 | 0.22 |
| Menthol | 0.2 |
| Polyquaternium-7 | 0.2 |
| Sodium hydroxide | 0.15 |
| Niacinamide | 0.1 |
| Zinc PCA | 0.1 |
| Dye/colorant | 0.1 |
| Citric acid | 0.06 |

### Example Formulation 1

Example Formulation 1 contains dihydroxyindole and caffeine as xanthine derivative.

| Component | Quantity in % by weight |
|---|---|
| Aqua [water] | ad 100 |
| Sodium lauryl ether sulfate | 8.0 |
| Sodium myreth sulfate | 2.5 |
| Laureth-2 | 2 |
| Panthenol | 1.7 |
| Sodium chloride | 1.65 |
| Disodium lauryl sulfosuccinate | 1.5 |
| Sodium lauroyl glutamate | 1.3 |
| Caffeine | 1.1 |
| PEG-120 methyl glucose dioleate | 0.75 |
| Wheat protein hydrolyzate | 0.7 |
| Dihydroxyindole | 0.49 |
| Allantoin | 0.4 |
| Citric acid | 0.38 |
| Sodium benzoate | 0.36 |
| Propylene glycol | 0.3 |
| Potassium sorbate | 0.3 |
| PEG-40 hydrogenated castor oil | 0.18 |
| Cocoglucoside | 0.18 |
| Glyceryl oleate | 0.17 |
| Polyquaternium-7 | 0.17 |
| Disodium EDTA | 0.15 |
| Zinc PCA | 0.1 |
| Niacinamide | 0.1 |
| Tocopherol | 0.06 |

### Example Formulation 2

Example Formulation 2 contains dihydroxyindole, caffeine as xanthine derivative and HC Blue 2 as direct dye.

| Component | Quantity in % by weight |
|---|---|
| Aqua [water] | ad 100 |
| Sodium lauryl ether sulfate | 11.5 |
| Sodium cocoamphoacetate | 3 |
| Cocamidopropyl betaine | 1.7 |
| Caffeine | 1.1 |
| Panthenol | 0.92 |
| Sodium chloride | 1 |
| Phenoxyethanol | 0.7 |
| Cocoglucoside | 0.5 |
| Glyceryl oleate | 0.5 |
| Phosphoric acid | 0.4 |
| PEG-120 methyl glucose dioleate | 0.3 |
| HC Blue 2 | 0.22 |
| Menthol | 0.2 |
| Polyquaternium-7 | 0.2 |
| Sodium hydroxide | 0.15 |
| Dihydroxyindole | 0.2 |
| Niacinamide | 0.1 |
| Zinc PCA | 0.1 |
| Dye/colorant | 0.1 |
| Citric acid | 0.06 |

### Example Formulation 3

Example Formulation 3 contains dihydroxyindole, caffeine as xanthine derivative and HC Blue 2 as direct dye.

| Component | Quantity in % by weight |
|---|---|
| Aqua [water] | ad 100 |
| Sodium lauryl ether sulfate | 9 |
| Sodium myreth sulfate | 3 |
| Sodium cocoamphoacetate | 1 |
| Laureth-2 | 2 |
| Disodium lauryl sulfosuccinate | 1 |
| Sodium lauroyl glutamate | 2 |
| Caffeine | 0.9 |
| Sodium chloride | 1 |
| Wheat protein hydrolyzate | 0.7 |
| Phosphoric acid | 0.3 |
| Sodium benzoate | 0.5 |
| Potassium sorbate | 0.2 |
| HC Blue 2 | 1 |
| Allantoin | 0.4 |
| PEG-40 hydrogenated castor oil | 0.4 |
| Polyquaternium-7 | 0.2 |
| Disodium EDTA | 0.2 |
| Dihydroxyindole | 0.3 |
| Tocopherol | 0.02 |

### Example Formulation 4

Example Formulation 4 contains dihydroxyindole, caffeine as xanthine derivative and HC Blue 2 as direct dye.

| Component | Quantity in % by weight |
|---|---|
| Aqua [water] | ad 100 |
| Sodium lauryl ether sulfate | 11 |
| Sodium myreth sulfate | 2 |
| Laureth-2 | 3 |
| Cocoamidopropyl betaine | 2 |
| Disodium lauryl sulfosuccinate | 1 |
| Sodium lauroyl glutamate | 1 |
| Panthenol | 0.5 |
| Caffeine | 1 |
| Sodium chloride | 0.9 |
| Phenoxyethanol | 0.5 |
| Wheat protein hydrolyzate | 0.5 |
| Cocoglucoside | 0.3 |
| Glyceryl oleate | 0.4 |
| Phosphoric acid | 0.25 |
| Sodium benzoate | 0.2 |
| PEG-120 methyl glucose dioleate | 0.1 |
| Potassium sorbate | 0.5 |
| HC Blue 2 | 0.8 |
| Allantoin | 0.1 |
| PEG-40 hydrogenated castor oil | 0.2 |
| Menthol | 0.2 |
| Sodium hydroxide | 0.1 |
| Disodium EDTA | 0.2 |
| Dihydroxyindole | 0.1 |
| Citric acid | 0.03 |
| Niacinamide | 0.2 |
| Zinc PCA | 0.2 |
| Dye/colorant | 0.1 |

### Example Formulation 5

Example Formulation 5 contains dihydroxyindole and caffeine as xanthine derivative.

| Component | Quantity in % by weight |
|---|---|
| Aqua [water] | ad 100 |
| Sodium lauryl ether sulfate | 12 |
| Sodium cocoamphoacetate | 2 |
| Cocoamidopropyl betaine | 2 |
| Panthenol | 1 |
| Caffeine | 1.1 |
| Sodium chloride | 0.9 |
| Phenoxyethanol | 0.8 |
| Cocoglucoside | 0.4 |
| Glyceryl oleate | 0.3 |
| Phosphoric acid | 0.3 |
| PEG-120 methyl glucose dioleate | 0.2 |
| Menthol | 0.2 |
| Polyquaternium-7 | 0.1 |
| Sodium hydroxide | 0.2 |
| Disodium EDTA | 0.1 |
| Dihydroxyindole | 0.2 |
| Citric acid | 0.06 |
| Niacinamide | 0.2 |
| Zinc PCA | 0.2 |
| Dye/colorant | 0.5 |

### Example Formulations 6-8

In contrast to Example Formulation 2, the additional dye for coloring the formulation was completely omitted in Example Formulations 6-8. Example Formulation 6 contains dihydroxyindole and caffeine as xanthine derivative, Example Formulation 7 contains dihydroxyindole, caffeine as xanthine derivative and HC Blue 2 as direct dye and Example Formulation 8 contains caffeine as xanthine derivative and HC Blue 2 as direct dye.

| | Example | Example | Example | Example |
|---|---|---|---|---|
| | Formulation 2 | Formulation 6 | Formulation 7 | Formulation 8 |
| | Quantity in % | Quantity in % | Quantity in % | Quantity in % |
| Component | by weight | by weight | by weight | by weight |
| Aqua [water] | ad 100 | ad 100 | ad 100 | ad 100 |
| Sodium lauryl ether sulfate | 11.5 | 11.5 | 11.5 | 11.5 |
| Sodium cocoamphoacetate | 3 | 3 | 3 | 3 |
| Cocamidopropyl betaine | 1.7 | 1.7 | 1.7 | 1.7 |
| Caffeine | 1.1 | 1.1 | 1.1 | 1.1 |
| Panthenol | 0.92 | 0.92 | 0.92 | 0.92 |
| Sodium chloride | 1 | 1 | 1 | 1 |
| Phenoxyethanol | 0.7 | 0.7 | 0.7 | 0.7 |
| Cocoglucoside | 0.5 | 0.5 | 0.5 | 0.5 |
| Glyceryl oleate | 0.5 | 0.5 | 0.5 | 0.5 |
| Phosphoric acid | 0.4 | 0.4 | 0.4 | 0.4 |
| PEG-120 methyl glucose dioleate | 0.3 | 0.3 | 0.3 | 0.3 |
| HC Blue 2 | 0.22 | — | 0.22 | 0.22 |
| Menthol | 0.2 | 0.2 | 0.2 | 0.2 |
| Polyquaternium-7 | 0.2 | 0.2 | 0.2 | 0.2 |
| Sodium hydroxide | 0.15 | 0.15 | 0.15 | 0.15 |
| Dihydroxyindole | 0.2 | 0.2 | 0.2 | - |
| Niacinamide | 0.1 | 0.1 | 0.1 | 0.1 |
| Zinc PCA | 0.1 | 0.1 | 0.1 | 0.1 |
| Dye/colorant | 0.1 | — | — | — |
| Citric acid | 0.06 | 0.06 | 0.06 | 0.06 |

### Neutral Formulation

The neutral formulation contains neither caffeine nor DHI nor HC Blue 2.

| Component | Quantity in % by weight |
|---|---|
| Aqua [water] | ad 100 |
| Sodium lauryl ether sulfate | 10 |
| Laureth-2 | 1.9 |
| Disodium lauryl sulfosuccinate | 1.5 |
| Sodium chloride | 1.3 |
| Sodium lauroyl glutamate | 1.3 |
| Panthenol | 1.2 |
| PEG-120 methyl glucose dioleate | 0.5 |
| Wheat protein hydrolyzate | 0.49 |
| Citric acid | 0.4 |
| Sodium citrate | 0.35 |
| Propylene glycol | 0.2 |
| Menthol | 0.2 |
| PEG-40 hydrogenated castor oil | 0.2 |
| Potassium sorbate | 0.19 |
| Polyquaternium-7 | 0.17 |
| Disodium EDTA | 0.11 |
| Sodium benzoate | 0.1 |
| Zinc PCA | 0.06 |
| Niacinamide | 0.055 |
| Tocopherol | 0.032 |
| Dye/colorant | 0.0005 |

### Application Examples

To evaluate the color performance in terms of quality, intensity and duration, the following washing tests (wash-on and wash-out tests) were carried out.

### Application Example 1

Kerling strands were subjected to a wash-on test with a shampoo containing 5,6-DHI, caffeine and HC Blue 2 (Example Formulation 2) and with a shampoo containing HC Blue 2 and caffeine (Comparative Formulation), respectively, in order to examine the color performance in terms of quality and intensity.

### Washing Protocol:

Moisten strands with lukewarm water for 30 seconds, brush out excess water between two fingers
Massage in a total of 5 g shampoo for 1 minute per 5 strands
Leave for a total of 5 minutes
Rinse with lukewarm water for 1 minute, then squeeze out in kitchen roll
Combing, spread out individually to air dry
The next day, scan strands with the Konica (Konica-Minolta Spectrophotometer CM600d, measuring geometry d/8°, Specular Component Excluded (SCE), standard illuminant D65, visual field) 10° (photograph sample strands every 5 washes); then repeat washing cycle

### Used Hair Strands:

Kerling white selected, adhesive braid 2 cm wide, 12 cm long, 3 g/pc

### Used Shampoos:

| | |
|---|---|
| Example Formulation 2 | Strands D1-D5 |
| Comparative Formulation | Strands E1-E5 |

For each product, 5 strands were used and 28 washes were carried out.

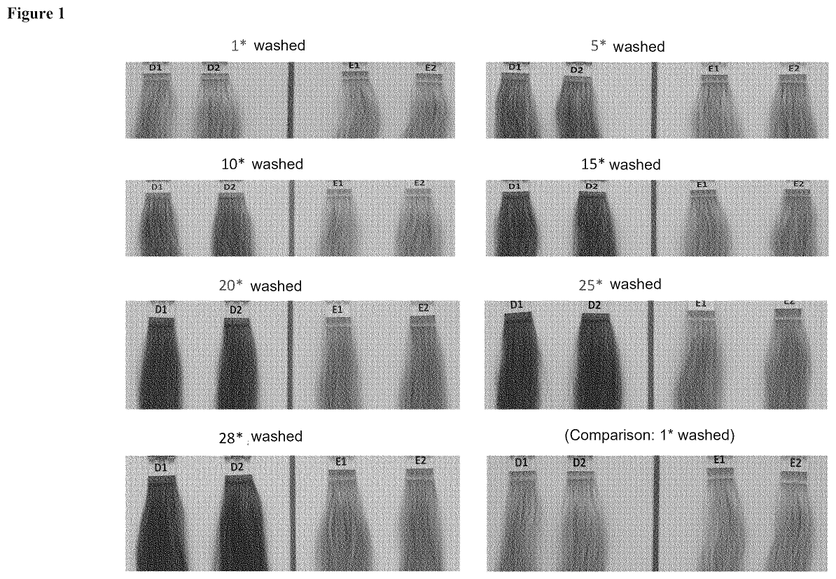

FIG. 1 shows a comparison of the strands D1/D2 treated with Example Formulation 2 with strands E1/E2 which were treated with the Comparative Formulation.

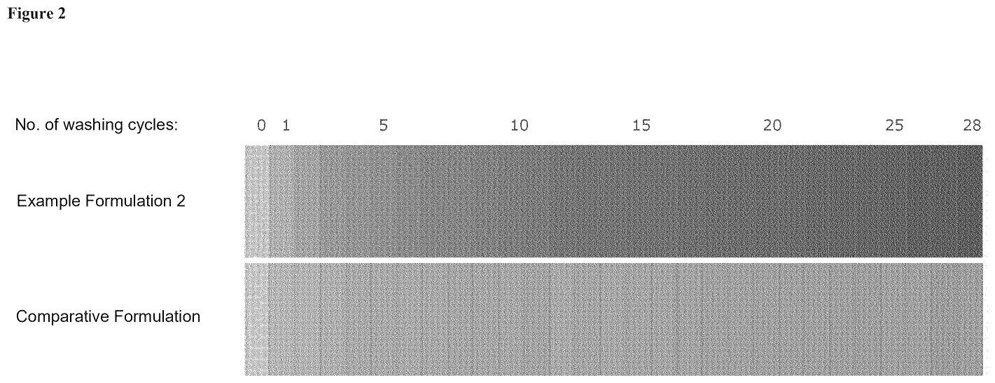

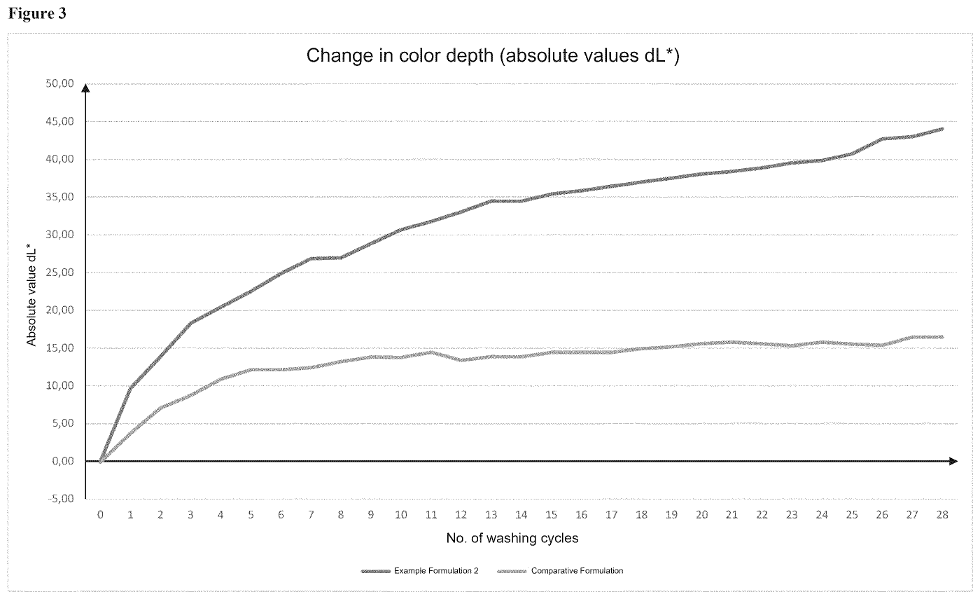

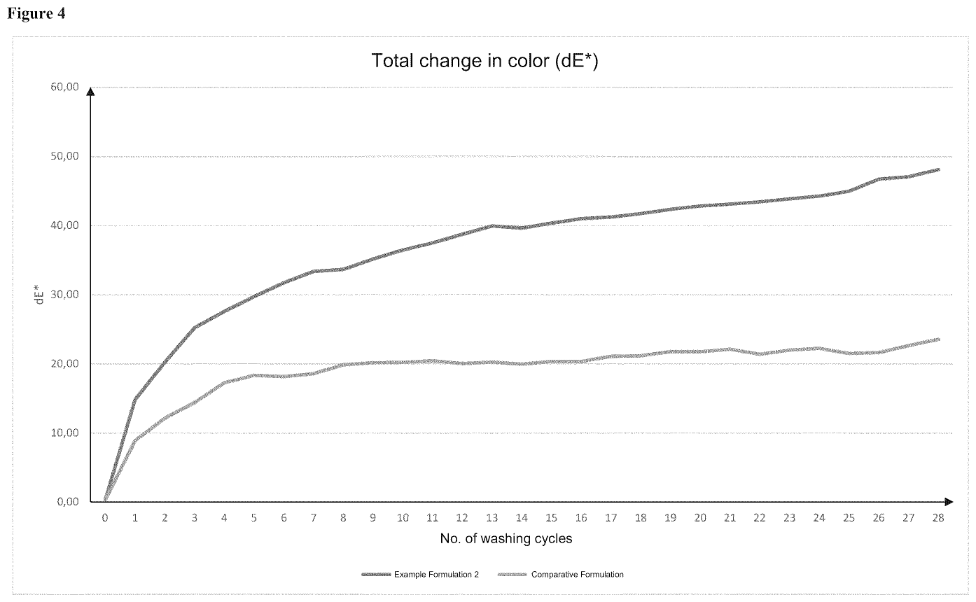

The comparison of the color preview for the two shampoos used is shown in FIG. 2. FIGS. 3 and 4 show the change in color depth as absolute values dL* (FIG. 3) and as total color change dE* (FIG. 4). The figures show the gradual build-up of the color effect with repeated use and the improved coloring result compared to the Comparative Formulation.

### Application Example 2

Kerling strands were subjected to a wash-on and wash-out test with a shampoo containing 5,6-DHI, caffeine and HC Blue 2 (Example Formulation 2) and with a shampoo containing HC Blue 2 and caffeine (Comparative Formulation), respectively, in order to examine the color performance in terms of quality, intensity and duration.

### Washing Protocol:

Moisten strands with lukewarm water for 30 seconds, brush out excess water between two fingers
Massage in a total of 5 g shampoo for 1 minute per 5 strands
Leave for a total of 5 minutes
Rinse with lukewarm water for 1 minute, then squeeze out in kitchen roll
Combing, spread out individually to air dry
The next day, scan strands with the Konica (Konica-Minolta Spectrophotometer CM600d, measuring geometry d/8°, Specular Component Excluded (SCE), standard illuminant D65, visual field) 10°, photograph; then repeat washing cycle

### Used Hair Strands:

Kerling white selected, adhesive braid 2 cm wide, 12 cm long

### Used Color Shampoos:

| | |
|---|---|
| Example Formulation 2 | Strands B1-B15 |
| Comparative Formulation | Strands C1-C15 |

The neutral formulation was used for washing out.

For each product, 15 strands were treated in portions of 5 pieces.

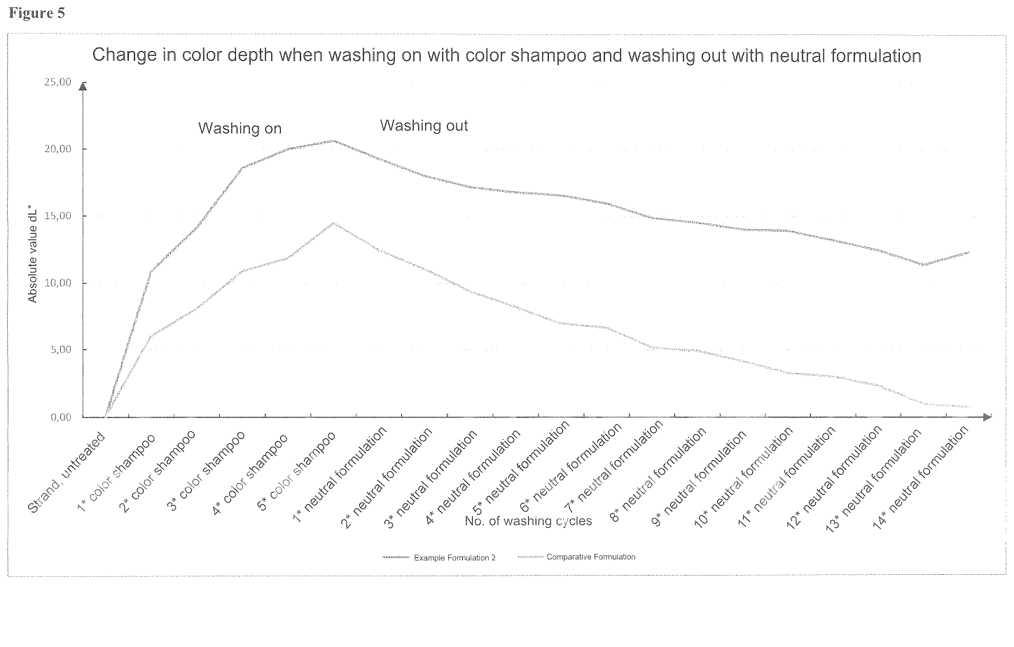

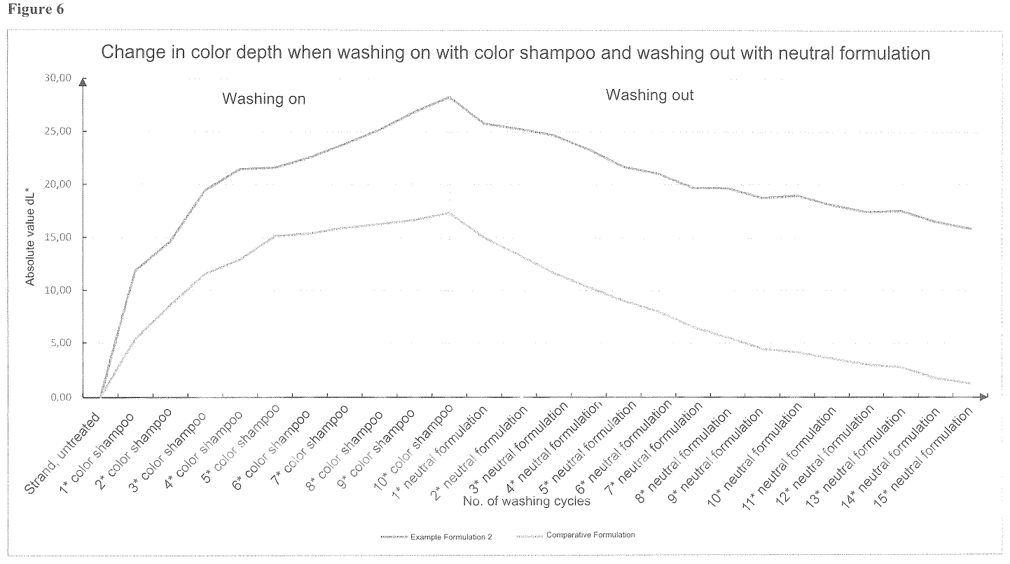

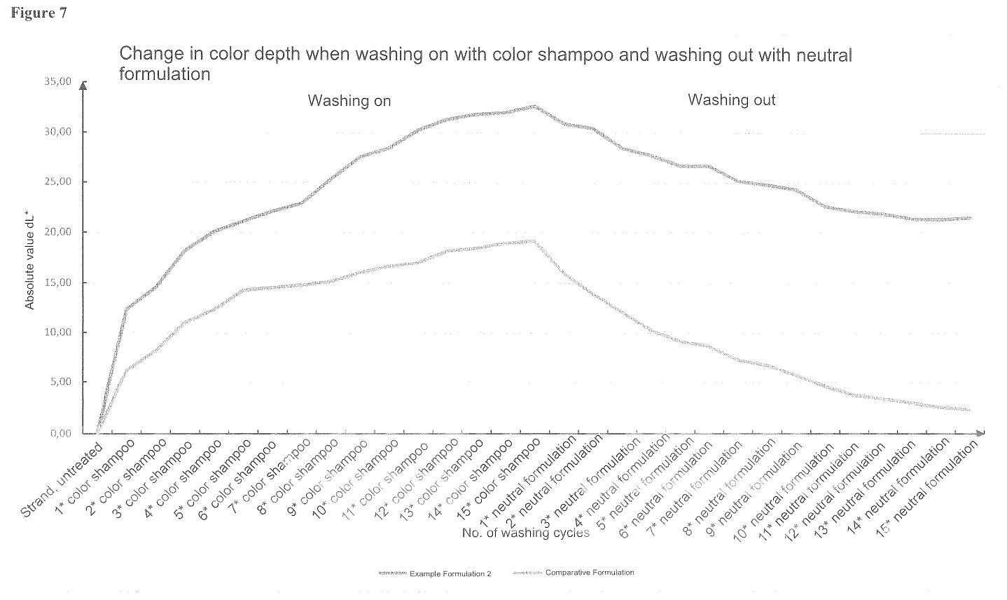

The color change was monitored throughout the subsequent wash-on and wash-out cycles:
5 washes each with color shampoo to wash on, followed by 14 washes with neutral formulation (FIG. 5)
10 washes each with color shampoo to wash on, followed by 15 washes with neutral formulation (FIG. 6)
15 washes each with color shampoo to wash on, followed by 15 washes with neutral formulation (FIG. 7).

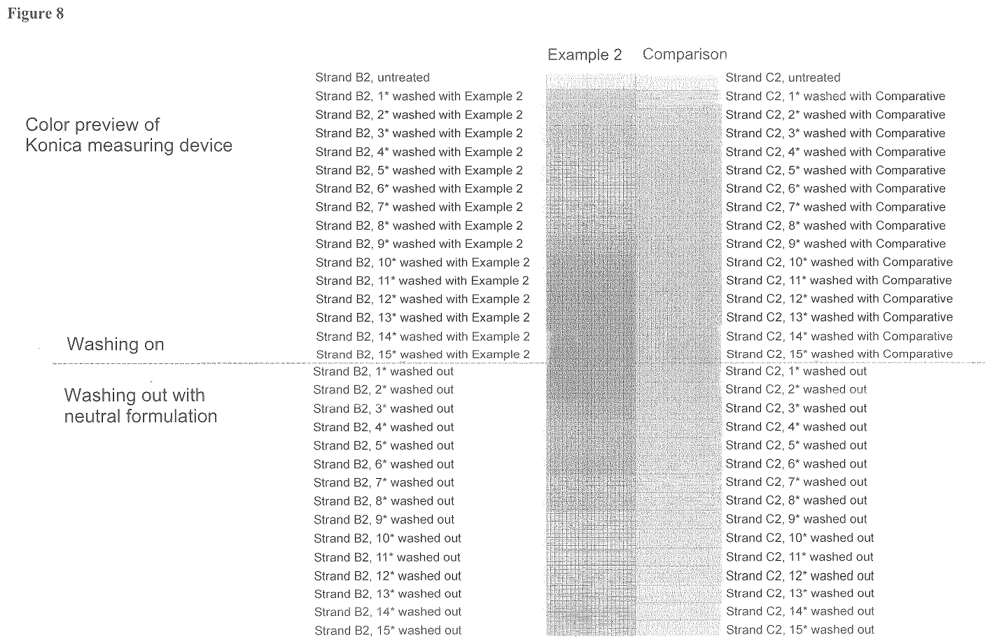

FIGS. 5-7 each show the change in color depth when washing on with color shampoo or washing out with neutral formulation according to the protocols given above. FIG. 8 shows the comparison of the color change for the two shampoos used on a single strand each at 15 wash-on cycles, followed by 15 wash-out cycles, measured with the Konica measuring device (Konica-Minolta Spectrophotometer CM600d, measuring geometry d/8°, Specular Component Excluded (SCE), standard illuminant D65, visual field) 10°. The figures show the gradual build-up of the color effect with repeated application and the improved staining result compared to the Comparative Formulation. After discontinuing the treatment with the shampoo according to the invention, the color effect also lasts longer than when using the Comparative Formulation.

### Application Example 3

White Virgin strands (Imhair company) were subjected to a wash-on test with a shampoo containing 5,6-DHI and caffeine (Example Formulation 1) and with a shampoo containing 5,6-DHI, caffeine and HC Blue 2 (Example Formulation 2), respectively, and with a shampoo containing HC Blue 2 and caffeine (Comparative Formulation), respectively, in order to examine the color performance in terms of quality and intensity.

### Washing Protocol:

Moisten strands with lukewarm water for 30 seconds, brush out excess water between two fingers
Massage in a total of 2 g shampoo for 30 seconds per 3 strands
Leave for a total of 5 minutes
Rinse with lukewarm water for 1 minute
Combing, air drying
The next day, scan strands with the Konica, photograph; then repeat washing cycle

### Used Hair Strands:

White Virgin (Imhair company)

### Used Shampoos:

| | |
|---|---|
| Example Formulation 1 | Strands 1-3 |
| Example Formulation 2 | Strands 4-6 |
| Comparative Formulation | Strands 7-9 |

For each product, 3 strands each were used and 15 washing cycles each were carried out.

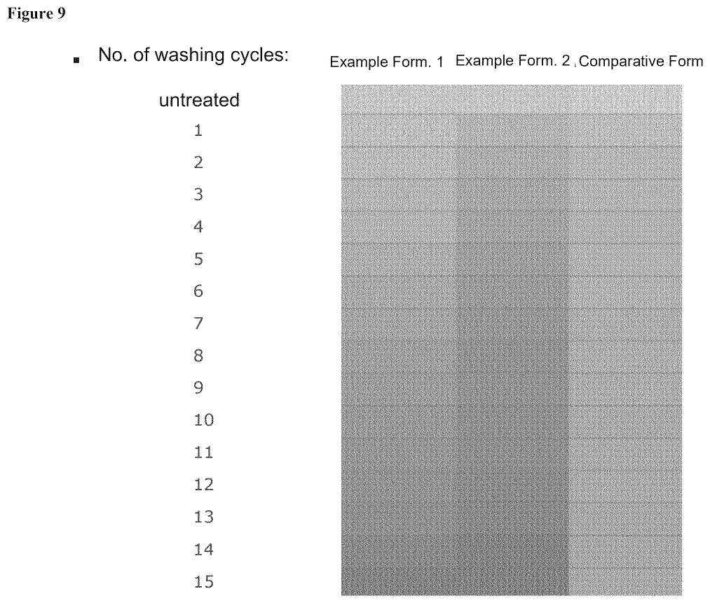

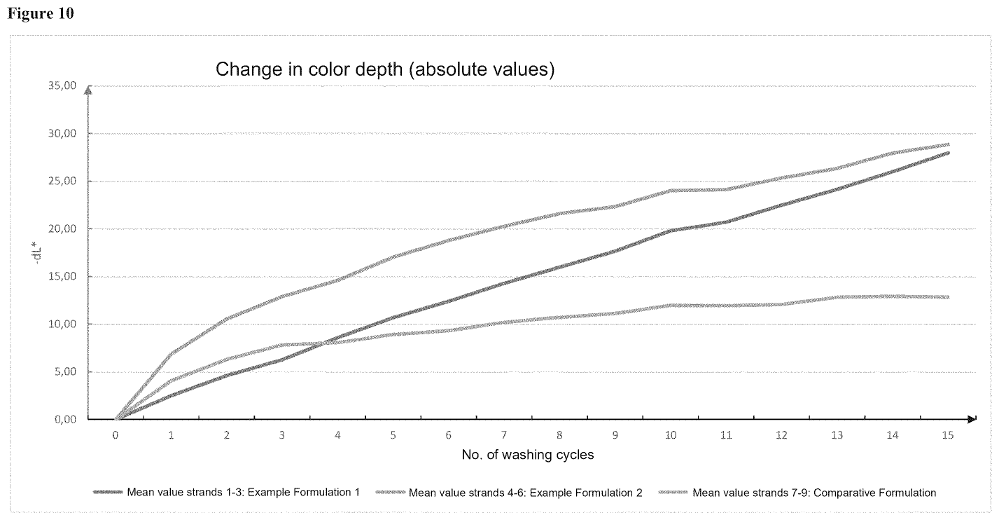

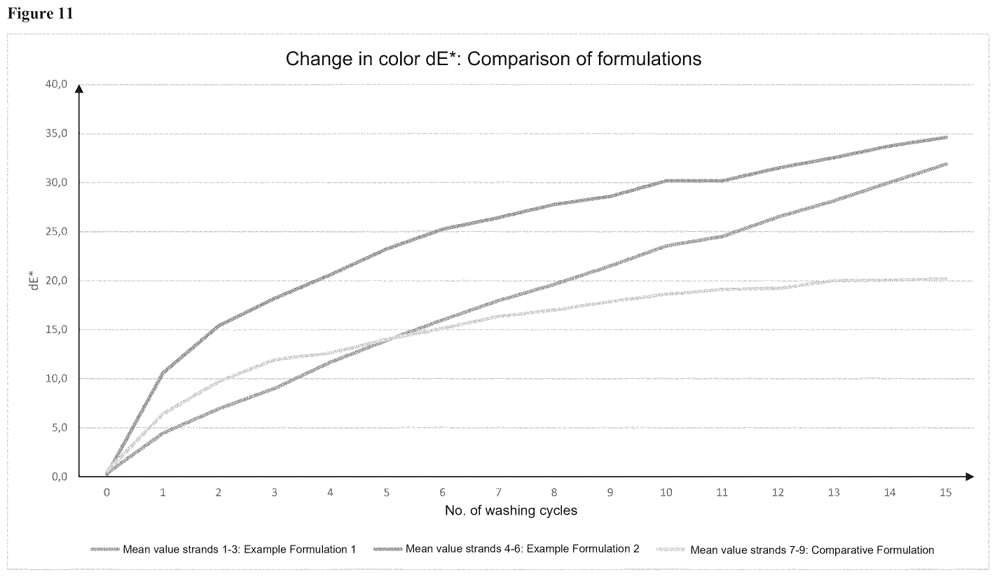

The comparison of the color preview for the shampoos used is shown for a single strand of hair in FIG. 9. FIGS. 10 and 11 show the change in color depth as absolute values dL* (FIG. 10) and as total color change dE* (FIG. 11). The figures show the gradual build-up of the color effect with repeated application and the improved dyeing result compared to the Comparative Formulation.

### Application Example 4

To examine the dyed shade over time, the Kerling white and Imhair bleached strands were dyed for 15 min with 10 g of Example Formulation 2, rinsed for 1 min and air-dried for 1.5 h (residual moisture was then blow-dried). Measurements were taken on the day of treatment after 15 min exposure time (initial shade) and after 1, 2, 3, 8, 9, 10, 11, 14 and 15 days.

| | L*(D65) | a*(D65) | b*(D65) | dL*(D65) | da*(D65) | db*(D65) | dE*ab(D65) |
|---|---|---|---|---|---|---|---|
| Kerling strand, white | 76.58 | 2.15 | 21 | — | — | — | — |
| Initial shade | 62.32 | 1.58 | 5.02 | −14.27 | −0.57 | −15.98 | 21.43 |
| (after 15 min | | | | | | | |
| exposure time) | | | | | | | |
| read in after 1 d | 62.11 | 2.05 | 5.23 | −14.48 | −0.1 | −15.77 | 21.41 |
| read in after 2 d | 63.06 | 2.22 | 4.59 | −13.52 | 0.07 | −16.42 | 21.27 |
| read in after 3 d | 63.02 | 2.44 | 5.09 | −13.56 | 0.29 | −15.91 | 20.91 |
| read in after 8 d | 62.95 | 2.53 | 5.57 | −13.64 | 0.38 | −15.44 | 20.6 |
| read in after 9 d | 62.86 | 2.35 | 5.5 | −13.73 | 0.21 | −15.51 | 20.71 |
| read in after 10 d | 62.63 | 2.57 | 5.87 | −13.95 | 0.42 | −15.13 | 20.58 |
| read in after 11 d | 62.59 | 2.61 | 5.47 | −14 | 0.46 | −15.53 | 20.91 |
| read in after 14 d | 63.16 | 3.1 | 6.34 | −13.43 | 0.95 | −14.67 | 19.91 |
| read in after 15 d | 63.59 | 3.16 | 6.26 | −12.99 | 1.02 | −14.74 | 19.68 |

| | L*(D65) | a*(D65) | b*(D65) | dL*(D65) | da*(D65) | db*(D65) | dE*ab(D65) |
|---|---|---|---|---|---|---|---|
| Imhair strand, | 76.9 | 3.5 | 24.61 | — | — | — | — |
| bleached | | | | | | | |
| Initial shade | 51.51 | 1.01 | 2.73 | −25.38 | −2.49 | −21.88 | 33.61 |
| (after 15 min | | | | | | | |
| exposure time) | | | | | | | |
| read in after 1 d | 51.71 | 1.6 | 2.8 | −25.19 | −1.9 | −21.82 | 33.38 |
| read in after 2 d | 52.11 | 1.61 | 2.79 | −24.78 | −1.89 | −21.82 | 33.08 |
| read in after 3 d | 51.57 | 2.06 | 3.23 | −25.33 | −1.43 | −21.38 | 33.17 |
| read in after 8 d | 52.21 | 2.22 | 3.78 | −24.68 | −1.28 | −20.84 | 32.33 |
| read in after 9 d | 51.98 | 2.13 | 3.84 | −24.92 | −1.36 | −20.78 | 32.47 |
| read in after 10 d | 52 | 2.29 | 4.16 | −24.9 | −1.21 | −20.46 | 32.24 |
| read in after 11 d | 52.11 | 2.29 | 4.16 | −24.79 | −1.21 | −20.46 | 32.16 |
| read in after 14 d | 52.69 | 2.65 | 4.86 | −24.21 | −0.84 | −19.75 | 31.25 |
| read in after 15 d | 53.23 | 2.68 | 5.01 | −23.67 | −0.82 | −19.61 | 30.74 |

The measured values show a—also visually perceptible—shift in shade from the original graphite to a more natural, more brownish color.

### Application Example 5

Kerling strands were subjected to a wash-on test with a shampoo containing 5,6-DHI and caffeine (Example Formulation 6) and with a shampoo containing 5,6-DHI, caffeine and HC Blue 2 (Example Formulation 7), respectively, and with a shampoo containing HC Blue 2 and caffeine (Example Formulation 8), respectively, in order to examine the color performance in terms of quality and intensity.

### Washing Protocol:

Moisten 5 strands with lukewarm water for 30 s, squeeze out excess water between two fingers
Massage in a total of 5 g shampoo for 1 min per 5 strands
Leave for a total of 5 minutes
Rinse with lukewarm water for 1 minute, then squeeze out with kitchen roll
Comb, spread out individually to air dry
The next day, scan strands with the Konica (Konica-Minolta Spectrophotometer CM600d, measuring geometry d/8°, Specular Component Excluded (SCE), standard illuminant D65, visual field) 10°, photograph; then repeat washing cycle

### Used Hair Strands:

Kerling white selected, adhesive braid 2 cm wide, 12 cm long, 3 g/pc,

### Used Coloring Shampoos:

| | |
|---|---|
| Example Formulation 6 | strands DHI-A to E |
| Example Formulation 7 | strands Combi-A to E |
| Example Formulation 8 | strands Blue-A to E |

5 strands were used per product, 15 washes were carried out in each case.

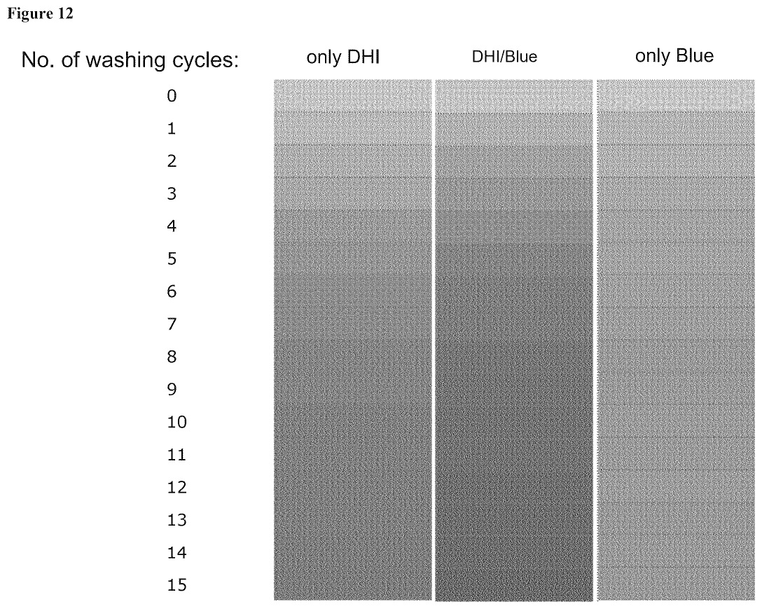

The comparison of the color preview for the shampoos used is shown for a single strand of hair in FIG. 12.

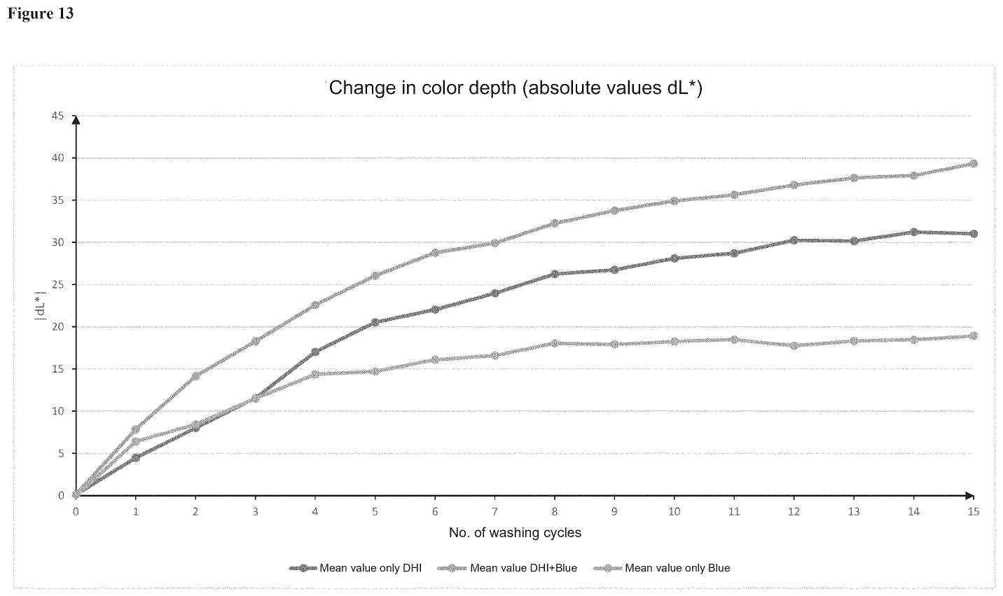

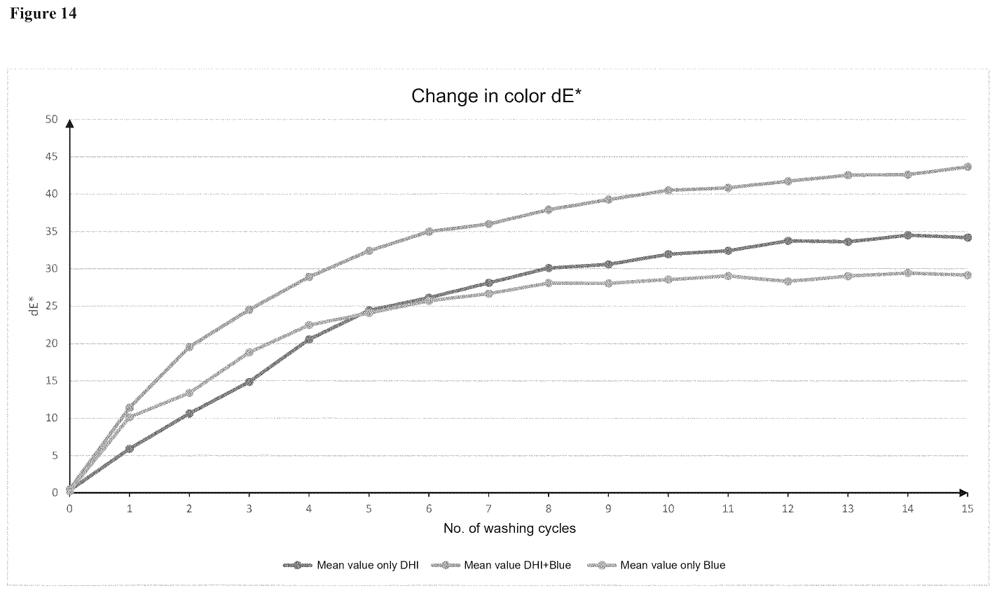

FIGS. 13 and 14 show the change in color depth as absolute values dL* (FIG. 13) and as total color change dE* (FIG. 14).

In FIGS. 12-14, the term “DHI only” refers to tests with a shampoo containing 5,6-DHI and caffeine (Example Formulation 6), the term “DHI/Blue 2” refers to tests with a shampoo containing 5,6-DHI, caffeine and HC Blue 2 (Example Formulation 7) and the designation “Blue 2 only” refers to tests with a shampoo containing HC Blue 2 and caffeine (Example Formulation 8).

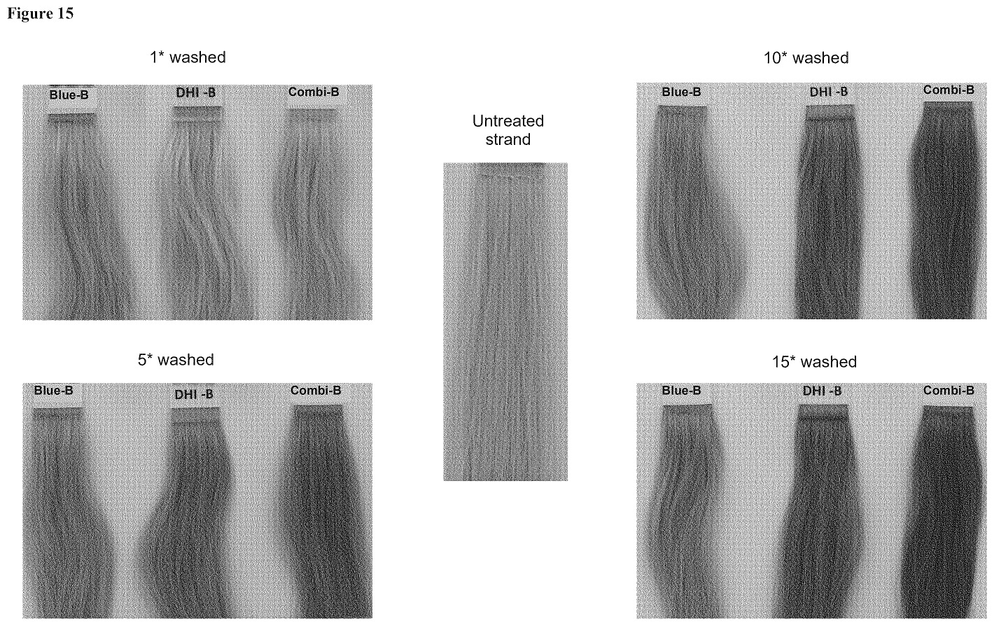

FIG. 15 shows a comparison of the strands washed with Example Formulation 6 (“DHI-B”), with Example Formulation 7 (“Combi-B”) and with Example Formulation 8 (“Blue-B”) using strand B as an example.

The figures show the gradual build-up of the color effect with repeated application and the improved coloring result compared to a Comparative Formulation only containing HC Blue 2 and caffeine (Example Formulation 8).

1. A cosmetic formulation comprising a dye precursor selected from the group consisting of indoline derivatives and indole derivatives, and a xanthine derivative as bioactive ingredient.

2. The cosmetic formulation according to claim 1, wherein the dye precursor is an indole derivative.

3. The cosmetic formulation according to claim 1, wherein the dye precursor is a derivative of 5,6-dihydroxyindole of formula (I), 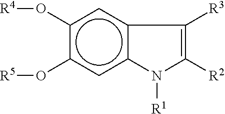
wherein, independently of each other,
R¹ is hydrogen, a C₁-C₄-alkyl group or a C₁-C₄-hydroxyalkyl group,
R² is hydrogen or a —COOH group, whereby the —COOH group can also be present as a salt with a physiologically tolerated cation,
R³ is hydrogen or a C₁-C₄-alkyl group,
R⁴ is hydrogen, a C₁-C₄-alkyl group, an amino group or a group —CO—R⁶ in which R⁶ is a C₁-C₄-alkyl group, and
R⁵ is one of the groups mentioned under R⁴,
or a physiologically tolerated salt of these compounds with an organic or inorganic acid.

4. The cosmetic formulation according to claim 3, wherein the compound of formula (I) is selected from 5,6-dihydroxyindole, N-methyl-5,6-dihydroxyindole and physiologically tolerated salts thereof.

5. The cosmetic formulation according to claim 1, wherein the dye precursor is an indoline derivative.

6. The cosmetic formulation according to claim 1, wherein the dye precursor is a derivative of 5,6-dihydroxyindoline of formula (II), 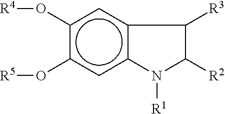
wherein, independently of each other,
R¹ is hydrogen, a C₁-C₄-alkyl group or a C₁-C₄-hydroxyalkyl group,
R² is hydrogen or a —COOH group, whereby the —COOH group can also be present as a salt with a physiologically tolerated cation,
R³ is hydrogen or a C₁-C₄-alkyl group,
R⁴ is hydrogen, a C₁-C₄-alkyl group, an amino group or a group —CO—R⁶ in which R⁶ denotes a C₁-C₄-alkyl group, and
R⁵ is one of the groups mentioned under R⁴,
or a physiologically tolerated salt of these compounds with an organic or inorganic acid.

7. The cosmetic formulation according to claim 6, wherein the compound of formula (II) is selected from 5,6-dihydroxyindoline, N-methyl-5,6-dihydroxyindoline and physiologically tolerated salts thereof.

8. The cosmetic formulation according to claim 1, wherein the xanthine derivative is caffeine.

9. The cosmetic formulation according to claim 1, further comprising at least one direct dye.

10. The cosmetic formulation according to claim 9, wherein the direct dye is selected from the group consisting of 2-amino-6-chloro-4-nitrophenol, 1,4-bis-(β-hydroxyethyl)-amino-2-nitrobenzene, Basic Yellow 57, Basic Red 76, Basic Brown 16, Basic Brown17, Basic Blue 99, HC Blue 2, HC Blue 12, HC Blue 16 and Acid Violet 43, preferably HC Blue 2.

11. Use of the cosmetic formulation according to claim 1 for the repigmentation of keratin fibers, in particular human hair.

12. The use according to claim 11 for the repigmentation of human hair, wherein the formulation is distributed in wet or dry hair and is rinsed out again after a dwell time of between 1 and 30 minutes.

13. The use according to claim 11 for the repigmentation of human hair, wherein the hair is permanently colored in a slow and gentle manner and at the same time the keratinocytes in the hair roots are stimulated so that they produce more natural pigment again.

14. A method for repigmenting keratin fibers, in particular human hair, wherein the cosmetic formulation according to claim 1 is applied to the hair and then the coloring is carried out.
